SEQUENCE LISTING (18 sequences)

SEQ ID NO: 1 - protein, 9 aa, Oryza sativa
CDALSVLVR

SEQ ID NO: 2 - protein, 8 aa, Oryza sativa
QLLEPCCR

SEQ ID NO: 3 - protein, 18 aa, Oryza sativa
CNLQHTGFFGCPMFGGGM

SEQ ID NO: 4 - protein, 12 aa, Oryza sativa
LSEALGVSSQVA

SEQ ID NO: 5 - protein, 8 aa, Oryza sativa
LQAFEPIR

SEQ ID NO: 6 - protein, 8 aa, Oryza sativa
DFLLAGNK

SEQ ID NO: 7 - protein, 12 aa, Oryza sativa
SQAGTTEFFDVS

SEQ ID NO: 8 - protein, 12 aa, Oryza sativa
VEPQQCSIFAAG

SEQ ID NO: 9 - protein, 11 aa, Oryza sativa
VIQPQGLLVPR

SEQ ID NO: 10 - protein, 11 aa, Ambrosia trifida
  misc_feature (9)..(9): Xaa can be any naturally occurring amino acid
LCEKPSLTXSG

SEQ ID NO: 11 - protein, 8 aa, Ambrosia trifida
CIEWEGAK

SEQ ID NO: 12 - protein, 9 aa, Ambrosia trifida
VDHIVGEEK

SEQ ID NO: 13 - protein, 10 aa, Ambrosia trifida
GDFPVFYVTK

SEQ ID NO: 14 - protein, 12 aa, Ambrosia trifida
QIAQGDELVFNY

SEQ ID NO: 15 - protein, 11 aa, Ambrosia trifida
QIVQGDELVFK

SEQ ID NO: 16 - protein, 7 aa, Dermatophagoides pteronyssinus
YTWNVPK

SEQ ID NO: 17 - protein, 12 aa, Dermatophagoides pteronyssinus
GKPFQLEAVFEA

SEQ ID NO: 18 - protein, 10 aa, Dermatophagoides pteronyssinus
FIDCGHNEVK

(19) United States
(12) Patent Application Publication
(10) Pub. No.: US 2006/0228769 A1
(43) Pub. Date: Oct. 12, 2006

(54) Method of detecting allergen protein

(75) Inventors: Hiroyuki Yano, Niigata (JP); Shigeru Kuroda, Ibaraki (JP)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: Inc., Admn. Agncy, Nat. Agr. & Bio-orntd Rsrch Org, Ibaraki (JP)

(21) Appl. No.: 10/524,757

(22) PCT Filed: Jul. 8, 2003
(86) PCT No.: PCT/JP03/08668

§ 371 Date: Feb. 11, 2005

(30) Foreign Application Priority Data
Aug. 13, 2002 (JP) 2002-236048

(51) Int. Cl. C12Q 1/26; G01N 33/00

(52) U.S. Cl. 435/25; 436/86

(57) ABSTRACT

The present invention relates to a method for detecting a protein having a disulfide bond or an allergen protein, comprising: protecting by chemically modifying a free SH group of a protein in a sample to be tested; cleaving a disulfide bond of the free SH group-protected protein to expose SH groups; and detecting the exposed SH groups.

### TECHNICAL FIELD

The present invention relates to a method for detecting proteins, and particularly relates to a method for detecting allergen proteins.

### BACKGROUND ART

Allergen proteins are broadly present in our living environment. Allergen proteins show harmful effects resulting in food allergies, house dust allergies, pollenosis, and the like, and occasionally induce fatal symptoms, for example, of anaphylaxis. To elucidate the allergy onset mechanism and to develop a protective method against allergies, it is an important object to screen for allergen proteins comprehensively.

As a conventional screening method for allergen proteins, a detection method based on the immunoblotting method, which involves transferring proteins to membranes such as nitrocellulose or PVDF, reacting the proteins with IgE antibodies in the blood serum of an allergy patient, and then detecting the antibodies binding to the proteins, is employed (e.g., Weiss, W., et al., Electrophoresis, 18, 826-833 (1997)). When this method is employed, allergen proteins present in a very small quantity are easily lost upon transfer to membranes, indicating that some important allergen proteins may be overlooked and remain undetected. Hence, a method whereby allergen proteins can be detected without requiring any blotting procedures is desired.

In the meantime, as a general method for detecting proteins, a method that involves subjecting a sample containing proteins to two-dimensional electrophoresis (O'Farrell, P. H. et al, Journal of Biological Chemistry, 250, pp 4007-4021 (1975)) and visualizing proteins by staining gel using, for example, silver staining, dye staining (Coomassie staining), negative staining, or fluorescent staining, is conventionally employed.

Furthermore, a method that is also known involves previously reducing proteins in a sample, fluorescently labeling the proteins using monobromobimane, subjecting the labeled proteins to two-dimensional electrophoresis and visualizing fluorescence signals, thereby detecting the proteins with high sensitivity (Urwin, V. E., and Jackson, P., Anal. Biochem. 209, 57-62 (1993)).

However, there are no known methods whereby allergen proteins can be detected with high sensitivity.

### DISCLOSURE OF THE INVENTION

This application claims the priority of Japanese Patent Application No. 2002-236048, the disclosure in the specification/drawings of which is incorporated herein.

An object of the present invention is to provide a method for detecting a protein having a disulfide bond or an allergen protein with high sensitivity.

As a result of intensive studies to achieve the above object, we have developed a method for detecting a protein having a disulfide bond by protecting a free SH group of a protein in a sample to be tested, cleaving a disulfide bond of the protein to expose SH groups that had formed the bond, and then detecting the exposed SH groups. By the use of this method, we have further succeeded in detecting an allergen protein contained in a sample to be tested.

The present invention completed based on these results is as follows.
[1] A method for detecting a protein having a disulfide bond, comprising: protecting by chemical modification a free SH group of a protein in a sample to be tested; cleaving a disulfide bond of the free SH group-protected protein to expose SH groups; and detecting the exposed SH groups.

In this method, the exposed SH groups are preferably detected by reacting the exposed SH groups with an SH group-labeling substance and detecting the labeled SH groups. In this case, prior to detection of the labeled SH groups, proteins in a sample to be tested are preferably separated by two-dimensional electrophoresis.

In this method, it is particularly preferred to chemically modify a free SH group by alkylation with iodoacetamide and to use monobromobimane as an SH group-labeling substance.
[2] A method for detecting an allergen protein, by utilizing the method for detecting a protein having a disulfide bond according to [1] above.

Preferred examples of a sample to be tested include protein extracts from seeds of gramineous plants, pollens, mites, and house dust.
[3] A kit for detecting a protein having a disulfide bond or an allergen protein, containing an SH group-protecting agent and an SH group-detecting substance.
[4] A kit for detecting a protein having a disulfide bond or an allergen protein, containing iodoacetamide and monobromobimane.

The kits of [3] and [4] may further contain a reducing agent.

The method of the present invention can be typically implemented via, but are not limited to, for example, the following steps of (A) to (C). FIG. 1 schematically shows these steps.

(A) A step of reacting a protein in a sample to be tested with an SH group-protecting agent. In the step, free SH groups of the protein are chemically modified so as to lose their reactivity and come into a state of being protected (FIG. 1[A]).

(B) A step of reacting the free SH group-protected protein with a reducing agent. Thus, the disulfide bonds (shown as “S-S” in FIG. 1) of the protein are cleaved, and then the SH groups that had formed the disulfide bonds are exposed (FIG. 1[B]).

(C) A step of reacting the protein having the exposed SH groups with an SH group-labeling substance, thereby labeling only such exposed SH groups of the protein (FIG. 1[C]). Subsequently, by detection of the labeled SH groups, the protein having the exposed SH groups is detected. The protein detected in this manner is identified as a protein having disulfide bonds.

According to the method of the present invention, a protein having a disulfide bond can be specifically detected with high sensitivity. Furthermore, when such the method for detecting a protein having a disulfide bond is applied for detection of an allergen protein, the allergen protein in a sample to be tested can be efficiently detected. According to the method for detecting an allergen protein of the present invention, allergen proteins present in a very small quantity that has been difficult to detect by conventional screening methods for allergen proteins can also be detected with high sensitivity.

The present invention is described in detail as follows.

1. Definition of Basic Terms as Used Herein

In this specification, the following terms are used according to the meanings as defined below.
(1) “Protein” principally means a polypeptide chain or a peptide chain that is generated via condensation between amino acids. However, “protein” in the present invention encompasses not only simple proteins from which only amino acids are generated by hydrolysis, but also encompasses conjugated proteins from which amino acids and organic substances other than amino acids are generated by hydrolysis (e.g., nucleoproteins, glycoproteins, lipoproteins, phosphoproteins, and chromoproteins) and inducible proteins (e.g., gelatine, peptone, and proteose). These proteins may be further modified enzymatically or chemically.
(2) “Protein having a disulfide bond” means a protein wherein a disulfide bond is formed within the protein molecule or between protein molecules under nonreducing conditions. The disulfide bond may be a disulfide bond formed in vivo in the protein or may be a disulfide bond that is not formed in vivo.
(3) “Allergen” means an antigenic substance that causes an allergic reaction in humans or mammals. “Allergen protein” means a protein that can cause an allergic reaction in humans or mammals (i.e., a protein that acts as an allergen). An allergen protein may be a full-length protein produced by transcription and translation from a gene or may be a fragment thereof. In addition, “allergenicity” of a substance means ability to induce an allergic reaction in humans or mammals.
(4) “SH group” means a sulfhydryl group of a protein. Generally, this group may also be referred to as a thiol group or a mercapto group.
   2. Method for Detecting a Protein Having a Disulfide Bond

(1) Sample to be Tested

In this specification, a sample to which the method of the present invention is applied is referred to as a “sample to be tested.” As a sample to be tested, as long as it is suspected of containing proteins having disulfide bonds or allergen proteins, any sample can be used. The sample to be tested may contain one kind of protein or several kinds of proteins. The sample to be tested may also contain unisolated proteins or previously isolated proteins. The sample to be tested may be composed of only previously isolated proteins.

Examples of a sample to be tested include foods, medicines, medical materials, cosmetics, textiles, construction materials, samples for environmental testing (e.g., air, water, and soil samples), plant samples, animal samples, allergen candidate substances, known allergens, known allergen proteins, and research samples (e.g., proteins predicted to have disulfide bonds formed therein). Specific examples include, but are not limited to, protein extracts prepared from, for example, fish, meat, dairy products (e.g., milk, yogurt, and cheese), egg-containing products (e.g., mayonnaise, cakes, and noodles), prepared foods, seasonings, spices, nutritional supplements, wool products, feather products, house dust, mites (e.g., Dermatophagoides farinae and Dermatophagoides pteronyssinus), pollens (e.g., cedar pollens and ragweed pollens), food allergens (e.g., seeds of gramineous plants such as rice and wheat, buckwheat, soybean, albumen, and milk), allergens derived from animals (e.g., dogs, cats, mice, rats, horses, and cattle), allergens derived from plants (e.g., peanut and sumac), insect allergens, parasite allergens, or mold allergens. Further examples of a sample to be tested include purified proteins such as ovalbumin, ovomucoid, β-lactoglobulin, α-casein, mite antigens (e.g., Dermatophagoides farinae antigen), bovine serum albumin, trypsin/amylase inhibitors, glutelin, α-globulin, glycinin, isolated natural occurring proteins, recombinant proteins, proteins having altered natural amino acid sequences, synthetic proteins, and allergen candidate proteins. These samples to be tested may be used alone or may be combined and used in a single sample.

(2) Protection of a Free SH Group of a Protein in a Sample to be Tested

In this method, first free SH groups of proteins contained in the sample to be tested described in the above subsection 2-(1) are protected by chemical modification, so as to cause the free SH groups to lose their reactivity. “Free SH group” in this specification means an SH group in a protein, which does not participate in a disulfide bond under conditions that enable disulfide bond formation (e.g., nonreducing conditions such as neutral or acid conditions).

In the method of the present invention, to protect free SH groups of proteins contained in a sample to be tested, an SH group-protecting agent is added to the sample to be tested and is caused to react with the proteins. Reaction conditions when the SH group-protecting agent is added are not particularly limited, as long as they are nonreducing conditions. It is preferable to adjust conditions to those that are appropriate for an SH group-protecting agent to be used.

In the method of the present invention, beginning with protection of free SH groups of proteins allows the detection of only disulfide bond-forming SH groups without detecting such free SH groups, when SH groups are detected subsequent to the cleavage of disulfide bonds.

In this specification, a substance that protects free SH groups of proteins by chemical modification, so as to avoid reaction against the free SH groups is referred to as an “SH group-protecting agent.” Specific examples of “chemically modifying a free SH group” caused by such an SH group-protecting agent include mercaptide formation, alkylation, and oxidation accompanying disulfide exchange of the SH groups.

In the present invention, as an SH group-protecting agent, any substances that chemically modify SH groups to protect them as described above can be used. Specific examples of an SH group-protecting agent include SH reagents, which are used as protectively modifying agents of SH groups in proteins, and SH-blocking reagents, which react with SH groups in proteins to inhibit the reactivity.

Specific examples of an SH group-protecting agent include, but are not limited to, those described in the following (1) to (4):
(1) a mercaptide-forming agents, such as a heavy metal (e.g., mercury, silver, cadmium, lead, or copper) and a heavy metal compound (e.g., p-mercuribenzoic acid (PMB), p-chloromercuribenzoic acid (PCMB), p-mercuribenzenesulfonic acid (PMBS), phenylmercuric acetate (PMA), ethylmercury, 4-chloromercuri-4′-dimethylaminobenzene, 4-chloromercuriphenylazo-β-naphthol, salyrganic acid, or S-mercuridansyl dye);
(2) an alkylating agent such as an alkyl halide (e.g., iodoacetic acid or iodoacetamide) and a maleimide derivative (e.g., N-ethylmaleimide (NEM));
(3) an oxidant such as 5,5′-dithiobis (2-nitrobenzoic acid) (DTNB, Ellman's reagent), 4,4-dithiopyridine, 2,2′(4,4′)-dipyridyldisulphide, tetrathionic acid (or tetrathionate), tetranitromethane, 2,6-dichlorophenolindophenol (DCIP), or oxidized glutathione; and
(4) diarsenic trioxide and an arsenite.

The SH group-protecting agent of the present invention preferably does not disturb reducing action against disulfide bonds. Where an SH group-protecting agent that may disturb the reducing action is used, after protection of free SH groups and before reduction treatment for disulfide bonds, the residual SH group-protecting agent is preferably removed from a sample to be tested by, e.g., desalting.

As an SH group-protecting agent in the present invention, an alkylating agent is preferably used, and iodoacetamide (IAA) is particularly preferably used.

(3) Cleavage of a Disulfide Bond

Subsequently disulfide bonds of proteins are cleaved, so as to expose SH groups that had formed disulfide bonds. The cleavage of disulfide bonds is preferably carried out by reducing the disulfide bonds using a reducing agent. For the purpose of cleaving disulfide bonds using a reducing agent, free SH groups of proteins in a sample to be tested are protected according to description in the above subsection 2-(2), and then the proteins are caused to react with the reducing agent. The reducing agent may be added to a sample to be tested after reaction between free SH groups of proteins and an SH group-protecting agent. However, as long as proteins react with a reducing agent after protection of free SH groups of the proteins, the reducing agent may be added to the sample to be tested before or at the same time as the step of reaction of the free SH groups of the proteins with the SH group-protecting agent. For example, by adding a reducing agent sealed in a soluble capsule to a sample to be tested together with the SH group-protecting agent, free SH groups are protected using an SH group-protecting agent and then disulfide bonds of proteins are cleaved by the reducing agent released into a reaction solution from the dissolved capsule.

As a “reducing agent” used for cleaving disulfide bonds in the present invention, any substance capable of cleaving disulfide bonds of proteins so as to expose SH groups can be used. Specific examples of a reducing agent include, but are not limited to, dithiothreitol (DTT), dithioerythritol (DTE), glutathione, thioredoxin, glutathione reductase, mercaptoethanol, tributylphosphine, NaBH₄, NADPH, thioglycolic acid, and NO (nitrogen monoxide).

(4) Detection of an Exposed SH Group

Subsequently, SH groups exposed by cleavage of disulfide bonds are detected. Detection of the exposed SH groups may be carried out using a known method that can be used for detecting or quantifying SH groups.

For example, by a known method for detecting SH groups using an SH reagent, exposed SH groups can be detected and/or quantified. Examples of such a method for detecting SH groups include an amperometric titration method that utilizes mercaptide formation of exposed SH groups with a mercaptide-forming agent, which is an SH reagent, and a method for quantifying SH groups, which involves reacting p-mercuribenzoic acid (PMB), N-ethylmaleimide (NEM), 5,5′-dithiobis (2-nitrobenzoic acid), or the like with exposed SH groups and measuring absorbance of the thus obtained product. Another example of an SH reagent that is preferably used is 4′4′-bis(dimethylamino)benzhydrol.

In another embodiment, exposed SH groups can be detected and/or quantified by reacting an SH group-binding substance (e.g., a solid phase capable of binding to SH groups) with proteins having exposed SH groups and then detecting the SH group-binding substance that has bound to the proteins. Alternatively, proteins having exposed SH groups can also be detected and/or quantified by separating proteins that have bound to an SH group-binding substance and detecting the proteins. For example, exposed SH groups are selectively bind to a solid phase (e.g., beads, filters, membranes, or columns) capable of binding to SH groups, only the proteins that have bound to the solid phase are separated from a sample to be tested, and then the separated proteins may be detected. Specific examples of a solid phase capable of binding to SH groups include glutathione columns (glutathione sepharose column manufactured by Pharmacia Corporation) and magnetic beads having maleimide groups introduced therein. By this method, an increase in mass resulting from binding of SH group-binding substances with the proteins is measured, and then quantification of disulfide bonds of the proteins and determination of the disulfide bond sites can be carried out based on the increased amount.

As a still another example, exposed SH groups can be detected and/or quantified by reacting an SH group-labeling substance with proteins having exposed SH groups and then detecting labeled SH groups. As used herein, “label” includes, but are not limited to, fluorescent labels, radioactive isotope labels, antibody labels, and enzyme labels.

When an SH group-labeling substance is used, the method for detecting labels is determined depending on the type of the label. When a label is a fluorescent label, the signal from the label may be detected by, for example, detecting emitted fluorescence from labeled proteins irradiated with ultraviolet light using a fluorescence detector. The fluorescence signal can be detected by measuring the amount of fluorescence based on an absorbance corresponding to the fluorescence wavelength using a spectrophotometer. Since fluorescence is generally high in quantitativity, the amount of disulfide bonds or the disulfide bond sites can be determined based on the quantitatively determined fluorescent value (measured value). Furthermore, when a label is a radioactive isotope label, the signal of the label can be detected by measuring radioactivity using a liquid scintillation counter or the like. Alternatively, to detect the signal of the label, visualization is carried out by autoradiography, and then radioactivity is detected. When a label is an antibody label, for example, the label can be detected by reacting an antigen specific to the antibody used for labeling with the antibody. Furthermore, when a label is an enzyme label, the label can be detected by adding a substrate of the enzyme to react with the enzyme, and then detecting color development reaction, fluorescence reaction, or the like resulting from the enzyme reaction. When the non-fluorescent labels are used, the amount of the signal of the label is quantitatively determined upon detection so that the amount of disulfide bonds or the binding sites can be estimated from the measured value.

In the method of the present invention, fluorescent labeling reagents for SH groups can be appropriately used as SH group-labeling substances. Specific examples of an SH group-labeling substance include, but are not limited to, monobromobimane, a benzofurazan (benzoxadiazole) derivative (e.g., 4-fluoro-7-sulfamoylbenzofurazan (ABD-F)), aziridine such as dansylaziridine, fluoresceinmercuric acetate (FMA), S-mercuri-N-dansyl dye (MDC), N-(iodoacetylaminoethyl)-5-naphthylamine-1-sulfonic acid (1,5-I-AEANS) and a 1,8-isomer thereof, 4-chloro-7-nitrobenzofurazan (4-chloro-7-nitrobenzo-2-oxa-1,3-diazole) (NBD-Cl), and N-substituted maleimide having fluorescent groups (e.g., 2-phenylbenzimidazole, fluorescein, various rhodamines, and cyanine dye) introduced therein (e.g., N-(7-dimethylamino-4-methylcoumarynyl) maleimide (DACM), N-[4-(6-dimethylamino-2-benzofuranyl)phenyl] maleimide, and cyanine dye-maleimide).

In this specification, substances that can be used for detecting and quantifying SH groups, such as the above described SH reagents, SH group-binding substances, and SH group-labeling substances are referred to as “SH group-detecting substances.”

An SH group-detecting substance used in the method of the present invention may be any substance that can be used as the SH group-protecting agent described in the above subsection 2-(2). However, in the method of the present invention, preferably an SH group-protecting agent and an SH group-detecting substance that are different from each other are used in a single detection system. Furthermore, to specifically detect exposed SH groups, for example, preferably by the use of a non-fluorescent reagent as an SH group-protecting agent and a fluorescent reagent as an SH group-detecting substance, detection is carried out using the SH group-detecting substance without detecting protective modification by the SH group-protecting agent.

In the method of the present invention, a combination of an SH group-protecting agent and an SH group-detecting substance that can be particularly preferably used is a combination of iodoacetamide as an SH group-protecting agent and monobromobimane as an SH group-detecting substance.

An SH group-detecting substance may be added to a sample to be tested simultaneously with, before, or after the addition of a reducing agent; or simultaneously with, before, or after the addition of an SH-group protecting agent, as long as the SH group-detecting substance is caused to react with proteins after the SH groups of the proteins are exposed as described above. An example of such a case is a case where an SH group-detecting substance is contained in a soluble capsule or the like, such an SH group-detecting substance is added together with an SH group-protecting agent to a sample to be tested for the SH group-protecting agent to chemically modify free SH groups, and then the SH group-detecting substance is released in a reaction solution. In such a case, the SH group-detecting substance and a reducing agent contained in a single capsule, the SH group-detecting substance and a reducing agent contained in separate capsules, or the SH group-detecting substance contained alone in a capsule may be used. In this specification, the meaning of “adding” an SH group-detecting substance to a sample to be tested includes not only mixing an SH group-detecting substance into a sample to be tested, but also bringing an SH group-detecting substance into contact with a sample to be tested in other various states. For example, the meaning of “adding” includes, where an SH group-detecting substance is a solid phase substance capable of binding to SH groups, placing the SH group-detecting substance in a sample to be tested or applying a sample to be tested onto the SH group-detecting substance.

An SH group-detecting substance that is used in the present invention is preferably a substance whose reaction with exposed SH groups is not disturbed in its coexistence with an SH group-protecting agent and/or a reducing agent. However, when an SH group-detecting substance whose reaction with SH groups is disturbed in its coexistence with an SH group-protecting agent and/or a reducing agent is used, the SH group-protecting agent and/or the reducing agent is removed by desalting, protein separation, or the like before the addition of the SH group-detecting substance. In addition, the SH group-detecting substance of the present invention may be added together with an auxiliary agent such as a coloring substrate, if necessary.

In the method of the present invention, detection of exposed SH groups using an SH group-detecting substance may be carried out by ordinary methods. When exposed SH groups are detected using such SH group-detecting substance, exposed SH groups are caused to react with an SH group-detecting substance, and then detection can be directly carried out for a sample to be tested containing a number of proteins. However, after the reaction of exposed SH groups with an SH group-detecting substance, a sample to be tested may be previously separated by a conventionally known protein separation method, and then the exposed SH groups may be detected. For example, proteins in the above sample to be tested may be separated by a method known by persons skilled in the art, such as one-dimensional electrophoresis, two-dimensional electrophoresis, high performance liquid chromatography (HPLC), column chromatography, or mass spectrometry. After separation of proteins by subjecting a sample to be tested to electrophoresis, detection may be carried out on the electrophoretic gel. After separation of proteins by subjecting sample to be tested to high performance liquid chromatography (HPLC), detection may be carried out in the eluted molecular weight fractions. In the method of the present invention, it is particularly preferable to separate proteins by two-dimensional electrophoresis and then carry out detection thereof. With the use of two-dimensional electrophoresis, there is an advantage in that not only are proteins isolated with high resolution and high accuracy, but also the following detection for one sample to be tested can be completed in a single detection.

In another embodiment, if a free SH group-detecting substance as well as exposed SH groups might be detected using an SH group-detecting substance, it is preferable to separate proteins in a sample to be tested from the free SH group-detecting substance before detection of the exposed SH groups. An example of such a case is the use as an SH group-detecting substance of an SH group-labeling substance emitting labeling signals by itself, such as a maleimide reagent having fluorescein introduced therein as a fluorescent group. Furthermore, when a substance (e.g., the N-substituted maleimide having 2-phenylbenzimidazol introduced therein as a fluorescent group) that is characterized in that it is non-fluorescent by itself but that its derivative generated by reacting with SH groups is fluorescent is used as an SH group-detecting substance, labeled SH groups can be selectively detected without separating proteins from the free SH group-detecting substance.

In another embodiment, for example, absorbance that appears at around 280 nm corresponding to a cyclic structure having intramolecular S-S bonds generated by reaction between dithiothreitol and disulfide bonds when dithiothreitol is used as a reducing agent to cleave disulfide bonds, is measured so as to detect and quantify exposed SH groups. With such a technique, exposed SH groups can also be detected through detecting or quantifying products generated by reduction reaction of disulfide bonds.

According to the method of the present invention, proteins having disulfide bonds can be identified by detecting exposed SH groups as described above. When exposed SH groups are detected from a sample to be tested by the method of the present invention, the sample to be tested contains proteins having disulfide bonds. Moreover, when exposed SH groups are detected in each protein separated by electrophoresis or the like, the protein has disulfide bonds.

3. Isolation and Identification of a Protein Having a Disulfide Bond

Subsequently, proteins having disulfide bonds can be isolated from a sample to be tested in which exposed SH groups have been detected. The proteins may be isolated by a method known by persons skilled in the art. For example, when detection of exposed SH groups has been carried out for electrophoretic gel wherein proteins in a sample to be tested have been separated, each relevant spot portion of the gel in which exposed SH groups have been detected is cut out, and then proteins are extracted from the gel. For example, when detection of exposed SH groups is carried out for a molecular weight fraction obtained by separating proteins in a sample to be tested by HPLC, if necessary, proteins contained in the fraction are further purified by a purification technique such as chromatography. If proteins contained in the fraction are sufficiently purified, the fraction may be directly used as a solution containing isolated proteins. Alternatively, a target protein may also be isolated by carrying out affinity column purification for the above sample to be tested or the above fraction using an antibody for a label used herein. In addition, when, for example, a solid phase substance capable of binding to SH groups is used as an SH group-detecting substance, exposed SH groups selectively bind to the solid phase capable of binding to SH groups (e.g., beads, filters, membranes, or columns), proteins having the SH groups are separated from the sample to be tested utilizing the binding, and then the proteins having the SH groups are dissociated from the solid phase, so that proteins having disulfide bonds can be obtained. The thus obtained proteins can also be isolated by further separation and purification into each protein fraction by a purification technique such as chromatography, if necessary. By the above isolation step, proteins having disulfide bonds can be isolated, and the molecular weights, the content of the proteins in the sample to be tested, or the like can also be determined.

In the method of the present invention, proteins isolated as described above can also be identified by further characterization. “To identify proteins” in the present invention means to classify proteins into a protein group of known proteins or a protein group belonging to the same class as that of known proteins.

In the present invention, to identify an isolated protein, the protein are characterized, and the thus revealed characteristics are compared with those of known proteins so as to find common characteristics between the protein and a known protein. “Common characteristics” in the present invention means characteristics that are identical to or have much in common with those of known proteins. For example, if a characteristic relates to an amino acid sequence, a “common characteristic” in this case would involve possession of an identical amino acid sequence or possession of an amino acid sequence sharing high homology with that of the protein used for comparison. Furthermore, “characterization” in the present invention means to carry out for an isolated protein determination of the molecular weight and/or isoelectric point, etc. of such protein based on the results of electrophoresis, determination of a part of or the entire amino acid sequence, search and determination of a gene or a cDNA sequence encoding such protein, mass spectrometry for such protein, and the like. To determine an amino acid sequence, it is preferable to partially degrade a protein using peptidase or the like and then determine the internal amino acid sequence by subjecting each fragment to the Edman degradation method. In the meantime, multiple databases containing collections of such characteristics of proteins are present (e.g., GenBank, PIR, PRF, EMBL, SwissProt, and PDBSTR). By searching for the characteristics of proteins as isolated in the present invention within these databases, known proteins having common characteristics thereto can be extracted from the databases. When a known protein having characteristics identical to those of the protein isolated in the present invention is extracted from these databases, the isolated protein is identified as the known protein. When a known protein having characteristics similar to those of the isolated protein in the present invention is extracted from these databases, the isolated protein is classified as a protein of the same class as that of the known protein.

As described above, proteins having disulfide bonds, which are isolated in the present invention, can be identified.

4. Another Embodiment Utilizing the Method for Detecting a Protein Having a Disulfide Bond

In the present invention, through the utilization of the above method for detecting proteins having disulfide bonds, the presence or the absence of disulfide bond formation and the disulfide bond formation sites can be shown for purified specific proteins contained in a sample to be tested.

In recent years, protein production using genetic recombination techniques is being actively carried out. Regarding protein production by genetic recombination techniques, a method using Escherichia coli as host cells is general. However, since it is known that disulfide bonds are not correctly formed in proteins produced in Escherichia coli, recombinant proteins are produced using other cultured cells, such as insect cells, according to circumstances. Disulfide bonds in proteins are greatly involved in three-dimensional structure formation of such proteins and have a large effect on the maintenance of protein activity. Thus, in protein production process using recombination methods, it is important for the production of recombinant proteins having desired activity to confirm whether or not natural occurring proteins corresponding to recombinant proteins to be produced have disulfide bonds and to identify disulfide bond formation sites of the natural occurring proteins. Accordingly, analysis of disulfide bonds of natural occurring proteins that uses the method for detecting a protein having a disulfide bond of the present invention, can be useful in the production of recombinant proteins.

5. Method for Detecting an Allergen Protein

As shown in the following examples, proteins having disulfide bonds detected by the above method were revealed to be allergen proteins. Hence, according to the present invention, allergen proteins in a sample to be tested can be detected utilizing the above method for detecting proteins having disulfide bonds. That allergen proteins can be detected by the method for detecting an allergen protein of the present invention is also in consistent with a report that disulfide bonds relate to the allergenicity of proteins (e.g., Huby, R. D., et al., Toxicological Sciences 55, 235-246). The method for detecting an allergen protein of the present invention is very highly sensitive compared with the conventional detection method carried out by the immunoblotting method using an IgE antibody.

The method for detecting an allergen protein of the present invention may be implemented with similar procedures as those of the above method for detecting proteins having disulfide bonds. Specifically this method may be carried out by the following steps a) to c) of:

a) protecting a free SH group of a protein by chemical modification;

b) cleaving a disulfide bond of the free SH group-protected protein to expose SH groups that had formed the disulfide bond; and

c) detecting the exposed SH groups,

wherein the protein having the exposed SH groups is identified as an allergen protein.

A sample to be tested that is appropriate for detecting allergen proteins with application of the method for detecting an allergen protein of the present invention is similar to those described concerning the above method for detecting proteins having disulfide bonds. It is more preferable to use a protein sample suspected of containing allergen proteins as the sample to be tested. Examples of a sample to be tested that is particularly preferably used include protein extracts from seeds of gramineous plants, house dust, pollens, mites, or the like.

To perform the method for detecting an allergen protein of the present invention, particularly preferred specific procedures are as follows:
(1) adding a non-fluorescent reagent, iodoacetamide, to a sample to be tested;
(2) further adding dithiothreitol as a reducing agent;
(3) further adding a fluorescent reagent, monobromobimane, and then subjecting the obtained reaction solution to two-dimensional electrophoresis for separation; and
(4) irradiating the resultant electrophoretic gel with ultraviolet light, and then detecting the obtained fluorescent spots as corresponding to allergen proteins.

This method particularly has the following advantages compared with conventional methods.

Allergen proteins present even in a very small quantity can be detected using a fluorescent label.

Separation by two-dimensional electrophoresis leads to higher analytical resolution of proteins.

No transfer to a membrane is needed and risk of losing proteins present in a very small quantity is low.

In another embodiment of the present invention, it is possible to determine by the utilization of the above method for detecting allergen proteins, whether or not a sample to be tested has allergenicity. In this case, if allergen proteins are detected in the sample to be tested, the sample to be tested is determined to have allergenicity.

In another embodiment, by the utilization of the above method for detecting allergen proteins, screening of allergen proteins from a sample to be tested can be carried out. In this case, allergen proteins contained in the sample to be tested can be screened for in an isolating step of allergen proteins detected in the sample to be tested. The isolation of proteins can be carried out by generally known methods as in the above isolation of proteins having disulfide bonds.

For the allergen proteins isolated as described above, identification via characterization can be carried out in a manner similar to that used for proteins having disulfide bonds. Specifically, the isolated allergen proteins are characterized, and then the thus shown characteristics are compared with those of known allergen proteins, so as to find common characteristics between the proteins and the known allergen proteins. Meanings of “common characteristics” and “characterization” are the same as described for the above proteins having disulfide bonds.

Information about the characteristics of allergen proteins is also available from databases or the like (e.g., GenBank, PIR, PRF, EMBL, SwissProt, PDBSTR, and the Farrp allergen database (http://www.allergenonline.com/)). For example, characteristics of allergen proteins isolated in the present invention are searched for within such databases, so that known allergen proteins having common characteristics can be extracted from the databases. When a known allergen protein having characteristics identical to those of the isolated allergen protein is extracted from the databases, the isolated allergen protein is identified as the known allergen protein. When a known allergen protein having characteristics similar to those of the isolated allergen protein is extracted from the databases, the isolated protein is classified as an allergen protein of the same class as that of the known allergen protein.

In identification of allergen proteins, the following methods are generally employed in the art. Specifically, an internal amino acid sequence (partial sequence) of a protein of interest is determined by the Edman degradation method, and then the partial sequence as a query sequence is searched for within amino acid sequence databases. If a partial sequence of an allergen protein that is completely identical to the partial sequence of the protein of interest is extracted from the databases, the protein of interest is identified as such allergen protein extracted from the databases. Similarly, if a partial sequence of an allergen protein having high homology with the partial sequence of the protein of interest is extracted from the databases, the protein is identified as an allergen protein belonging to the same class as that of the allergen protein extracted from the databases. In the present invention, it is also preferable to utilize such method in identification of an isolated allergen.

6. Embodiments in Addition to Detection Method

Any method for analyzing a protein as provided in the present invention utilizes the above “method for detecting a protein having a disulfide bond.” A kit containing an SH group-protecting agent and an SH group-detecting substance used for detecting proteins having disulfide bonds can be preferably used for implementing the method for detecting a protein having a disulfide bond according to the present invention, the method for detecting an allergen protein according to the present invention, and any method for analyzing a protein utilizing these methods. A kit for detecting a protein having a disulfide bond or an allergen protein, which contains an SH group-protecting agent and an SH group-detecting substance, is also encompassed in the scope of the present invention. Preferably, the kit contains iodoacetamide and monobromobimane and is intended for detecting a protein having a disulfide bond or an allergen protein. The kit may further contain a reducing agent, preferably dithiothreitol.

### BRIEF DESCRIPTION OF THE DRAWINGS

FIG. 1 schematically shows typical steps to be performed in the method for detecting a protein having a disulfide bond of the present invention. Proteins having disulfide bonds are shown on the left side and proteins having no disulfide bonds are shown on the right side. Each white circle (◯) denotes modification of a SH group with an SH group-protecting agent and each filled circle (●) denotes modification of a SH group with an SH group-labeling substance. [A] Reaction of free SH groups of proteins with the SH group-protecting agent. [B] Reaction of the free SH group-protected proteins with a reducing agent. [C] Reaction of the exposed SH groups of the proteins with the SH group-labeling substance.

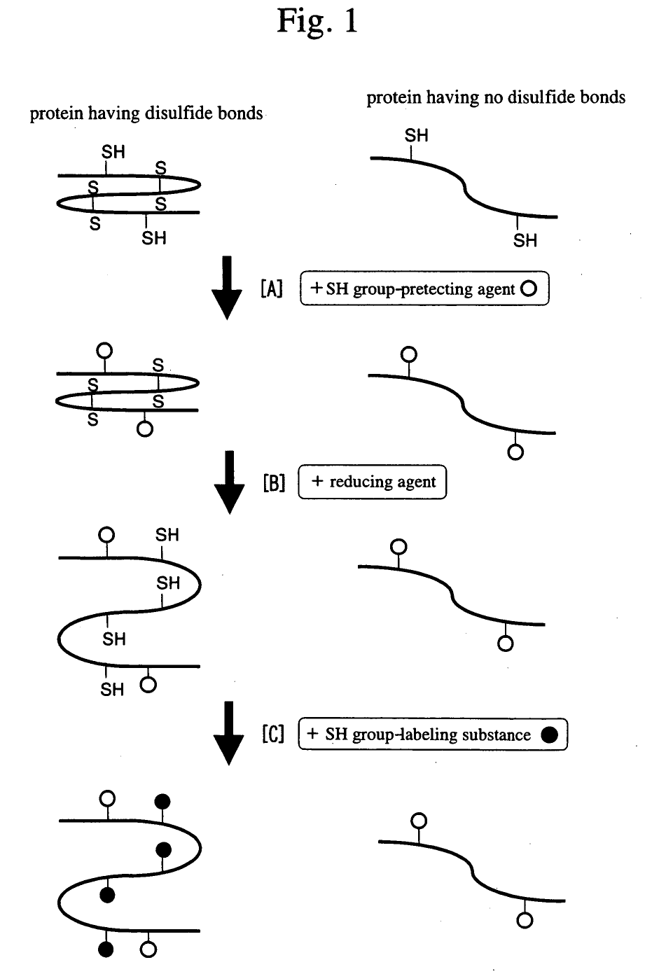

FIG. 2 shows photographs showing the results of two-dimensional electrophoresis obtained according to the method of the present invention using a rice seed extract as a sample to be tested. Photograph A shows the results of visualizing all the contained proteins by Coomassie blue staining. Photograph B shows the results of fluorescent detection of proteins wherein the SH groups were exposed according to the method of the present invention and then fluorescently labeled with monobromobimane. 1 to 8 indicate spots of the proteins whose internal amino acid sequences were determined.

FIG. 3 shows a photograph showing the results of two-dimensional electrophoresis obtained according to the method of the present invention using a pollen extract as a sample to be tested. Proteins fluorescently labeled with monobromobimane are indicated with white as a result of fluorescence detection. 1 to 6 indicate spots of the proteins whose internal amino acid sequences were determined.

FIG. 4 shows a photograph showing the results of two-dimensional electrophoresis obtained according to the method of the present invention using a mite extract as a sample to be tested. Proteins fluorescently labeled with monobromobimane are indicated with white as a result of fluorescence detection. 1 to 3 indicate spots of the proteins whose internal amino acid sequences were determined.

FIG. 5 shows a photograph showing the results of two-dimensional electrophoresis obtained according to the method of the present invention using a soybean trypsin inhibitor (STI) and myoglobin (Mg) as a sample to be tested. Photograph A shows the result of visualization of proteins contained in the sample to be tested by Coomasie blue staining. Photograph B shows the results of fluorescent detection of the proteins wherein the exposed SH groups were fluorescently labeled with monobromobimane.

### BEST MODE OF CARRYING OUT THE INVENTION

The present invention will be hereafter described in detail by referring to examples. However, these examples are not intended to limit the scope of the present invention.

### EXAMPLE 1

### Detection of Proteins Having Disulfide Bonds in a Rice Extract

20 g of rice seeds was ground using a mortar. The powder was put in 400 ml of 1 M sodium chloride solution and then agitated for 1 hour, so that proteins from the seeds were extracted in the buffer. After the solution was centrifuged at 14,000 g for 5 minutes to precipitate insoluble constituents, the supernatant was collected. The supernatant was desalted by dialysis and then freeze-dried. A 1/80 amount of the freeze-dried product (equivalent to 0.25 g of the rice seeds) was dissolved in a buffer for isoelectric focusing (8 M urea, 0.5% CHAPS, and 0.1% Bio-Lytes). lodoacetamide as an SH group-protecting agent was added (to a final concentration of 5 mM) to the solution, and then the solution was incubated at room temperature for 1 hour. Next, dithiothreitol as a reducing agent was added (to a final concentration of 5 mM) to the solution, and then the solution was incubated at room temperature for 1 hour. Furthermore, monobromobimane as an SH group-labeling substance was added (to a final concentration of 10 mM) to the solution, and then the solution was incubated at room temperature for 15 minutes.

The reaction solution obtained as described above was subjected to two-dimensional electrophoresis, thereby separating proteins in the reaction solution.

One-dimensional electrophoresis was performed using a PROTEAN IEF Cell System (Bio-Rad Laboratories Inc.) according to the manufacturer's instructions. Electrophoresis was carried out under conditions of an upper limit of 8,000 V and voltage-time integration value of 35,000 VH for 6 hours. After electrophoresis, the resultant gel was immersed in a buffer containing 62.5 mM Tris-HCl buffer, 5% mercaptoethanol, 2% SDS, and 5% sucrose for 10 minutes, and then subjected to two-dimensional electrophoresis.

Two-dimensional electrophoresis was carried out according to Laemmli's technique (Laemmli, U. K., Nature, 227, 680-685 (1970)). Acrylamide gel containing a 375 mM Tris/glycine buffer and having a concentration gradient ranging from 10% to 20% was used. As a running buffer, a 25 mM Tris/glycine buffer containing 0.1% SDS was used. The electrophoresis was carried out at 250 V for 3 hours.

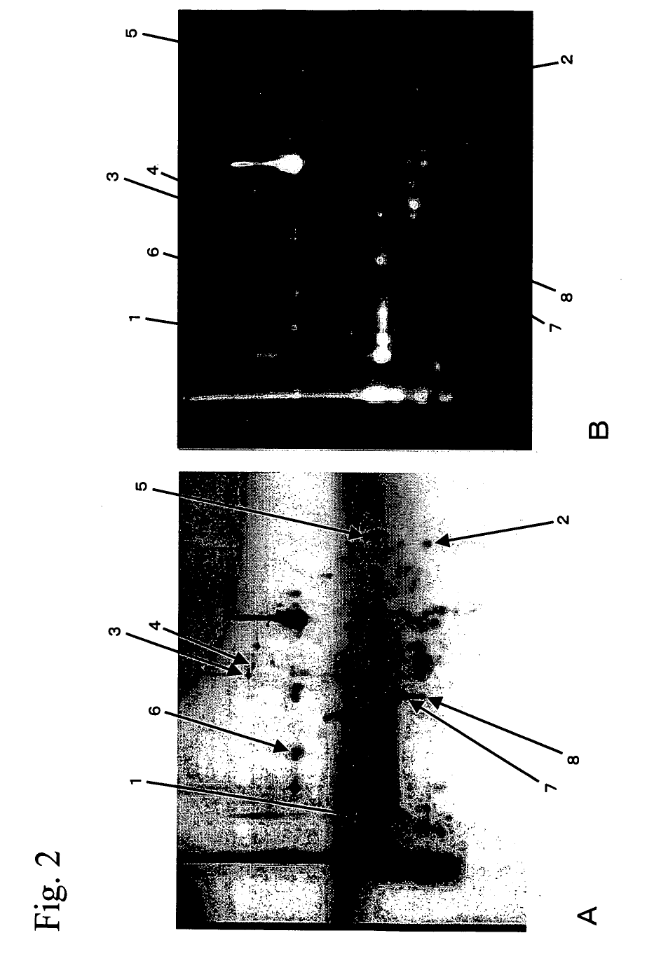

For the thus obtained electrophoretic gel, fluorescence signals were detected using a fluorescence detector (FAS-2525, TOYOBO). Fluorescently detected spots on the gel correspond to the proteins fluorescently labeled with monobromobimane (FIG. 2B). In FIG. 2B, fluorescent spots, for example, as indicated by 1 to 6 were found.

In addition, in this example, as a control experiment, all the proteins were detected by Coomassie blue staining (FIG. 2A). In FIG. 2A, for example, staining spots as indicated by 1 to 8 were found.

When FIG. 2A and FIG. 2B were compared, each of staining spots 1 to 6 in FIG. 2B was found to be at a position corresponding to each of staining spots 1 to 6 shown in FIG. 2A. That is, it was shown that proteins corresponding to spots 1 to 6 were proteins having disulfide bonds. These results suggested that the proteins present in spots 1 to 6 were allergen proteins.

On the other hand, for staining spots 7 and 8 in FIG. 2A, no fluorescent spots were found at corresponding positions in FIG. 2B. Thus, it was suggested that proteins corresponding to spots 7 and 8 in FIG. 2A were not allergen proteins.

### EXAMPLE 2

### Analysis of Allergen Proteins Contained in the Rice Extract

Spots 1 to 6 for which fluorescence signals were detected in FIG. 2B were excised from the gel and then digested within the gel using trypsin, so that the proteins in the spots were fragmented. After the fragmented proteins were subjected to HPLC for separation, the internal amino acid sequences were determined by the Edman method. The thus determined internal amino acid sequences for proteins in each spot are shown in Table 1. “C^{mBBr}” in the internal amino acid sequences shown in Table 1 denotes cysteine residues having SH groups labeled with monobromobimane. Next, a homology search was carried out for each of the internal amino acid sequences as a query sequence using the FASTA program and the BLAST program within amino acid sequence databases (GenBank and PIR). The search results showed that the internal amino acid sequence of the protein from each spot is identical to or has high homology with a partial amino acid sequence of a known allergen protein (Table 1).

TABLE 1

| | | | | |
|---|---|---|---|---|
| | | | | Number of |
| | | | | matching |
| Spot | | SEQ ID | Homologous protein | amino acids |
| No. | Internal amino acid sequence | NO: | extracted from databases | (%) |

| | | | | |
|---|---|---|---|---|
| 1 | C^{mBBr}DALSVLVR | SEQ ID | trypsin/amylase inhibitor | 7/9 |
| | | NO: 1 | | (77.8%) |
| | QLLEPC^{mBBr}C^{mBBr}R | SEQ ID | n.d. | |
| | | NO: 2 | | |
| | C^{mBBr}NLQHTGFFGC^{mBBr}PMFGGGM | SEQ ID | n.d. | |
| | | NO: 3 | | |
| 2 | LSEALGVSSQVA | SEQ ID | glutelin acidic chain | 12/12 |
| | | NO: 4 | | (100%) |
| 3 | LQAFEPIR | SEQ ID | glutelin acidic chain | 8/8 |
| | | NO: 5 | | (100%) |
| | DFLLAGNK | SEQ ID | glutelin acidic chain | 8/8 |
| | | NO: 6 | | (100%) |
| 4 | SQAGTTEFFDVS | SEQ ID | glutelin acidic chain | 12/12 |
| | | NO: 7 | | (100%) |
| 5 | VEPQQC^{mBBr}SIFAAG | SEQ ID | α-globulin | 12/12 |
| | | NO: 8 | | (100%) |
| 6 | VIQPQGLLVPR | SEQ ID | glutelin acidic chain | 11/11 |
| | | NO: 9 | | (100%) |

As shown in Table 1, the internal amino acid sequences of the proteins corresponding to spots 2 to 6 had 100% matches to partial amino acid sequences of known allergen proteins, which were extracted from the databases as proteins having high homology thereto. Accordingly, the proteins of spots 2 to 6 were identified as the known allergen proteins. In the meantime, one internal amino acid sequence of the protein of spot 1 had 77.8% homology with the partial amino acid sequence (SEQ ID NO: 1) of a trypsin/amylase inhibitor that is a known allergen protein, which was extracted from the databases as a protein having high homology thereto. Furthermore, for two other internal amino acid sequences (SEQ ID NOS: 2 and 3) of the protein corresponding to spot 1, no proteins having high homology thereto were extracted from the databases (shown with “n. d.” in Table 1). Accordingly, it was inferred that the protein of spot 1 belongs to the same allergen protein class as that of the trypsin/amylase inhibitor, but is an unknown allergen protein.

As described above, the proteins having disulfide bonds contained in the rice extract had been fluorescently detected according to the method of the present invention and could be identified as the unknown allergen protein (spot 1) belonging to the same class as that of the known allergen protein or as known allergen proteins (spots 2 to 6).

### EXAMPLE 3

### Detection of Proteins Having Disulfide Bonds in a Pollen Extract

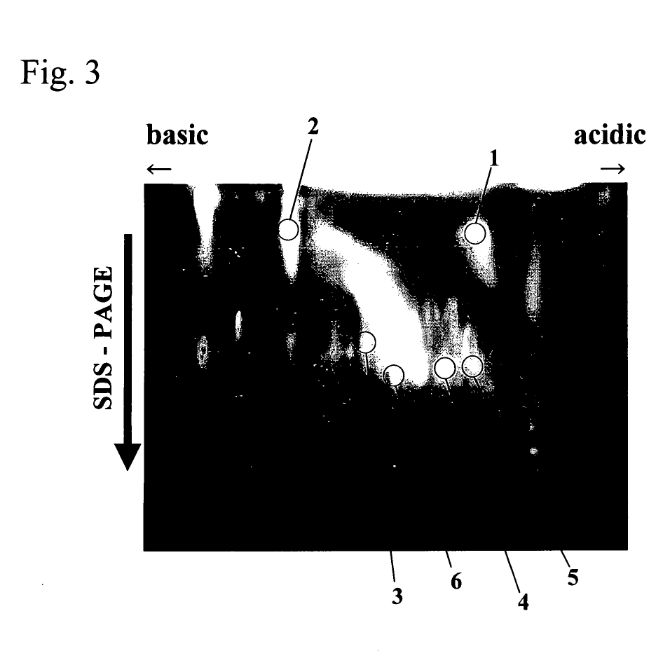

Ragweed (Ambrosia trifida) pollen was ground in a 100 mM Tris-HCl buffer (pH 8.0) containing 1 mM PASF and 1 mM EDTA using a mortar, and then the resultant was centrifuged at 14,000 g for 30 minutes. After centrifugation, the supernatant was collected, filtered through an Ultrafree-CL centrifugal filter (Millipore), and then desalted through a Microcon YM-10 centrifugal filter. The desalted residue was dissolved in a buffer for isoelectric focusing (8 M urea, 0.5% CHAPS, and 0.1% Bio-Lytes). lodoacetamide as an SH group-protecting agent was added (to a final concentration of 5 mM) to this solution, and then the solution was incubated at room temperature for 1 hour. Furthermore, dithiothreitol as a reducing agent was added (to a final concentration of 5 mM) to and mixed with the solution, and then the solution was incubated at room temperature for 1 hour. Furthermore, monobromobimane as an SH group-labeling substance was added (to a final concentration of 10 mM) to and mixed with the solution, and then the solution was incubated at room temperature for 15 minutes. The thus obtained reaction solution was subjected to two-dimensional electrophoresis, proteins in the reaction solution was separated in a manner similar to that in Example 1, and then fluorescence signals were detected using a fluorescence detector (FIG. 3).

### EXAMPLE 4

### Analysis of Allergen Proteins Contained in the Pollen Extract

Spots 1 to 6 for which fluorescence signals were detected in FIG. 3 were excised from the gel, and then the internal amino acid sequences of proteins were determined in a manner similar to that of Example 2. Subsequently, a homology search was carried out for the internal amino acid sequences in a manner similar to that of Example 2. The search results showed that the internal amino acid sequences of the proteins from each spot have high homology with the partial amino acid sequences of the known allergen proteins (Table 2).

TABLE 2

| | | | | Number of |
|---|---|---|---|---|
| | | | | matching |
| Spot | | SEQ ID | Homologous protein | amino acids |
| No. | Internal amino acid sequence | NO: | extracted from databases | (%) |
| 1 | LCEKPSLTXSG | SEQ ID | cysteine-rich antifungal protein | 9/11 |
| | (“X” denotes an | NO:10 | | (81.8%) |
| | unknown amino acid) | | | |
| 2 | CIEWEGAK | SEQ ID | anther-specific protein SF18 | 7/8 |
| | | NO:11 | | (87.5%) |
| 3 | VDHIVGEEK | SEQ ID | ABA-1 allergen | 6/9 |
| | | NO:12 | | (66.7%) |
| 4 | GDFPVFYVTK | SEQ ID | n.d. | — |
| | | NO:13 | | |
| 5 | QIAQGDELVFNY | SEQ ID | n.d. | — |
| | | NO:14 | | |
| 6 | QIVQGDELVFK | SEQ ID | n.d. | — |
| | | NO:15 | | |

As shown in Table 2, the internal amino acid sequence of a protein corresponding to spot 1 had 81.8% homology with a partial amino acid sequence of a cysteine-rich antifungal protein, which was extracted from the databases as a protein having high homology. Furthermore, the internal amino acid sequence of a protein corresponding to spot 2 had 87.5% homology with a partial amino acid sequence of anther-specific protein SF18, which was extracted from the databases as a protein having high homology. Both the cysteine-rich antifungal protein and anther-specific protein SF18 are known to belong to the defensin family that is allergen. Accordingly, it was shown that the protein of spot 1 and the protein of spot 2 are allergen proteins belonging to the same class as that of the cysteine-rich antifungal protein and the same class as that of anther-specific protein SF18, respectively. Moreover, the internal amino acid sequence of a protein corresponding to spot 3 had 66.7% homology with a partial amino acid sequence of allergen ABA-1, which was extracted from the databases as a protein having high homology thereto. Accordingly, it was shown that the protein of spot 3 is an allergen protein belonging to the same class as that of allergen ABA-1. For the internal amino acid sequences of proteins respectively corresponding to spots 4 to 6, no proteins having high homology thereto were extracted from the databases (shown with n. d. in Table 2). This suggested that the proteins of spots 4 to 6 are novel allergen proteins.

### EXAMPLE 5

### Detection of Proteins Having Disulfide Bonds in a Mite Extract

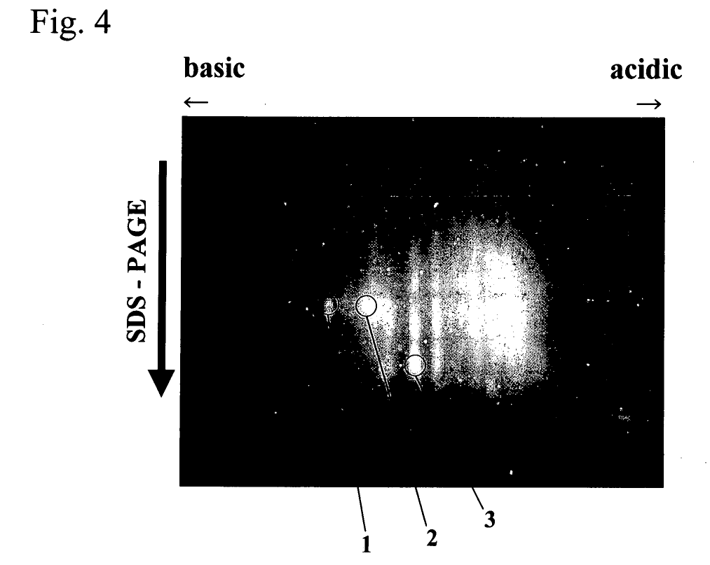

Dermatophagoides pteronyssinus was ground in a 100 mM Tris-HCl buffer (pH 8.0) containing 1 mM PASF and 1 mM EDTA using a mortar. The resultant was centrifuged at 14,000 g for 30 minutes. After centrifugation, the supernatant was collected, filtered through an Ultrafree CL centrifugal filter (Millipore), and then desalted through a Microcon YM-10 centrifugal filter. The desalted residue was dissolved in a buffer for isoelectric focusing (8 M urea, 0.5% CHAPS, and 0.1% Bio-Lytes). lodoacetamide as an SH group-protecting agent was added (to a final concentration of 5 mM) to this solution, and then the solution was incubated at room temperature for 1 hour. Furthermore, dithiothreitol as a reducing agent was added (to a final concentration of 5 mM), and then the solution was incubated at room temperature for 1 hour. Next, monobromobimane as an SH group-labeling substance was added (to a final concentration of 10 mM) to the solution, and then the solution was incubated for 15 minutes. The thus obtained reaction solution was subjected to two-dimensional electrophoresis, proteins in the reaction solution were separated in a manner similar to that in Example 1, and then fluorescence signals were detected using a fluorescence detector (FIG. 4).

### EXAMPLE 6

### Analysis of Allergen Proteins Contained in the Mite Extract

Spots 1 to 3 for which fluorescence signals were detected in FIG. 4 were excised from the gel, and then the internal amino acid sequences of proteins were determined in a manner similar to that of Example 2. Next, a homology search was carried out for the internal amino acid sequences in a manner similar to that of Example 2. The search results showed that the internal amino acid sequence of the protein from each spot has high homology with a partial amino acid sequence of each known allergen protein (Table 3).

TABLE 3

| | | | | Number of |
|---|---|---|---|---|
| | | | | matching |
| Spot | | SEQ ID | Homologous protein | amino acids |
| No. | Internal amino acid sequence | NO: | extracted from databases | (%) |
| 1 | YTWNVPK | SEQ ID | mite allergen DER P2 | 7/7 |
| | | NO:16 | | (100%) |
| 2 | GKPFQLEAVFEA | SEQ ID | mite allergen DER P2 | 12/12 |
| | | NO:17 | | (100%) |
| 3 | FIDCGHNEVK | SEQ ID | mite allergen GLY D2 | 8/10 |
| | | NO:18 | | (80%) |

As shown in Table 3, both internal amino acid sequences of the proteins corresponding to spots 1 and 2 had 100% homology with the partial amino acid sequences of mite allergen DER P2, a known allergen, which was extracted from the databases as a protein having high homology thereto. Furthermore, the internal amino acid sequence of the protein corresponding to spot 3 had 80% homology with the partial amino acid sequence of mite allergen GLY D2, a known allergen, which was extracted from the databases as a protein having high homology thereto. Accordingly, it was shown that the protein of spot 3 is an allergen protein belonging to the same class as that of mite allergen GLY D2.

As described above, the proteins having disulfide bonds contained in the mite extract had been fluorescently detected by the method of the present invention, and could be identified as known allergen proteins (spots 1 and 2) or an unknown allergen protein belonging to the same class as that of the known allergen protein (spot 3).

### EXAMPLE 7

### Detection Using Model Proteins

50 pmol of a soybean trypsin inhibitor (STI), the allergen protein having one free cysteine residue (that is, one free SH group) and two intramolecular disulfide bonds and 250 pmol of a Myoglobin (Mg) protein having no disulfide bonds and one free cysteine residue (that is, one free SH group), respectively, were mixed with 10 μl of a solution containing 62.5 mM Tris-HCl buffer (pH 6.8) and 2% SDS. lodoacetamide as an SH group-protecting agent was added (to a final concentration of 5 mM) to the solution, and then the solution was incubated at room temperature for 1 hour. Next, dithiothreitol as a reducing agent was added (to a final concentration of 5 mM) to the solution, and then solution was incubated at room temperature for 1 hour. Furthermore, monobromobimane as an SH group-labeling substance was added (to a final concentration of 10 mM) to the solution, and then the solution was incubated at room temperature for 15 minutes. The thus obtained reaction solution was subjected to SDS-PAGE to separate proteins in the reaction solution. SDS-PAGE was carried out according to Laemmli's method in a manner similar to that of Example 1.

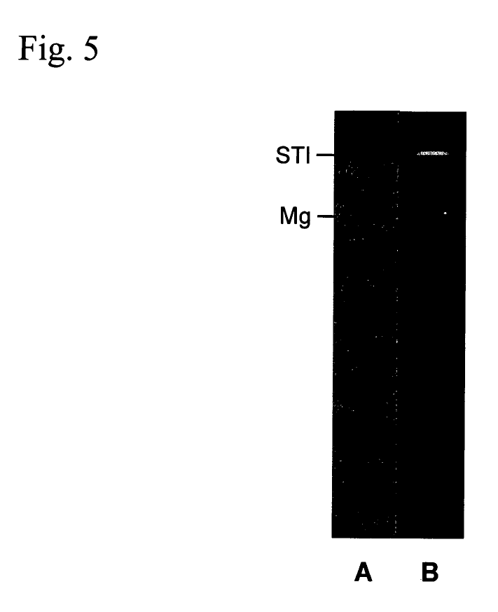

When all the proteins were detected by Coomassie blue staining, both STI and Mg proteins were detected in a way consistent with each protein amount (STI: 50 pmol and Mg: 250 pmol) (FIG. 5A). In contrast, when detection was carried out by labeling the proteins with monobromobimane, the fluorescent substance, only allergen STI having intramolecular disulfide bonds was detected, and Mg having only free cysteine residues and no disulfide bonds was not detected (FIG. 5B). It is considered that if not only disulfide bonds-forming SH groups but also SH groups of free cysteine residues are labeled together with monobromobimane by the method of the present invention, both types of SH groups should be detected with the same intensity of fluorescence. Hence, the result where only STI proteins were detected by the method of the present invention suggests that only SH groups that had formed intramolecular disulfide bonds in the STI proteins were labeled with monobromobimane.

From the above results, it was shown that proteins having disulfide bonds (that is, allergen proteins) can be specifically detected by the method of the present invention.

### INDUSTRIAL APPLICABILITY

The present invention provides a method for detecting with high sensitivity a protein having a disulfide bond in a sample to be tested and a method for detecting with high sensitivity an allergen protein in a sample to be tested. According to the method of the present invention, a protein having a disulfide bond or an allergen protein can not only be specifically detected, isolated, and/or identified, but can also be analyzed with high sensitivity and without losing proteins present in a very small quantity.

All publications, patents, and patent applications cited herein are incorporated herein by reference in their entirety.

1. A method for detecting a protein having a disulfide bond, comprising: protecting by chemically modifying a free SH group of a protein in a sample to be tested; cleaving a disulfide bond of the free SH group-protected protein to expose SH groups; and detecting the exposed SH groups.

2. The method of claim 1, wherein the exposed SH groups are detected by reacting the exposed SH groups with an SH group-labeling substance, and detecting the labeled SH groups.

3. The method of claim 2, wherein the protein in a sample to be tested is separated by two-dimensional electrophoresis before detection of the labeled SH groups.

4. The method of claim 2, wherein chemical modification is carried out by alkylation with iodoacetamide and the SH group-labeling substance is monobromobimane.

5. A method for detecting an allergen protein, comprising: protecting by chemically modifying a free SH group of a protein in a sample to be tested; cleaving a disulfide bond of the free SH group-protected protein to expose SH groups; and detecting the exposed SH groups.

6. The method of claim 5, wherein the exposed SH groups are detected by reacting the exposed SH groups with an SH group-labeling substance, and detecting the labeled SH groups.

7. The method of claim 6, wherein the protein in a sample to be tested is separated by two-dimensional electrophoresis before detection of the labeled SH groups.

8. The method of claim 6, wherein chemical modification is carried out by alkylation with iodoacetamide and the SH group-labeling substance is monobromobimane.

9. The method of claim 5, wherein the sample to be tested is a protein extract from seeds of gramineous plants, pollens, mites, or house dust.

10. A kit for detecting a protein having a disulfide bond or an allergen protein, containing an SH group-protecting agent and an SH group-detecting substance.

11. A kit for detecting a protein having a disulfide bond or an allergen protein, containing iodoacetamide and monobromobimane.

12. The kit of claim 10, further containing a reducing agent.

13. The method of claim 3, wherein chemical modification is carried out by alkylation with iodoacetamide and the SH group-labeling substance is monobromobimane.

14. The method of claim 6, wherein the sample to be tested is a protein extract from seeds of gramineous plants, pollens, mites, or house dust.

15. The method of claim 7, wherein chemical modification is carried out by alkylation with iodoacetamide and the SH group-labeling substance is monobromobimane.

16. The method of claim 7, wherein the sample to be tested is a protein extract from seeds of gramineous plants, pollens, mites, or house dust.

17. The method of claim 8, wherein the sample to be tested is a protein extract from seeds of gramineous plants, pollens, mites, or house dust.

18. The kit of claim 11, further containing a reducing agent.
